(19) United States
(12) Patent Application Publication
(10) Pub. No.: US 2017/0114091 A1
(43) Pub. Date: Apr. 27, 2017

(54) RESOLUBILIZATION OF PROTEIN CRYSTALS AT LOW PH

(71) Applicant: NOVOZYMES A/S, Bagsvaerd (DK)

(72) Inventors: Poul Erik Pedersen, Farum (DK); Jon Martin Persson, Bjaerred (SE); Esben Peter Friis, Herlev (DK)

(73) Assignee: NOVOZYMES A/S, Bagsvaerd (DK)

(21) Appl. No.: 15/128,724

(22) PCT Filed: Mar. 30, 2015
(86) PCT No.: PCT/EP2015/056921

§ 371 Date: Sep. 23, 2016

(30) Foreign Application Priority Data
Mar. 28, 2014 (EP) 14162436.1

(51) Int. Cl. C07K 1/14; C12N 9/54; C12N 9/36

(52) U.S. Cl. CPC C07K 1/145; C12N 9/2462; C12Y 302/01017; C12N 9/54; C12Y 304/21062

(57) ABSTRACT

A method for purifying a protein product prepared in a fermentation process where the protein of interest is present in solid, crystalline or amorphous form in the fermentation container is disclosed, where the pH of the fermentation broth is adjusted to a low pH whereby the protein of interest dissolves and can be efficiently separated from insoluble.

### REFERENCE TO A SEQUENCE LISTING

This application contains a Sequence Listing in computer readable form, which is incorporated herein by reference.

### FIELD OF THE INVENTION

The present invention relates to the field of protein purification, in particular to the field of protein purification of proteins prepared by a fermentation process.

### BACKGROUND OF THE INVENTION

An important part of an industrial protein production is the purification of the desired protein from the reminder of the production mixture wherein the protein has been generated.

In the fermentation industry proteins are in general produced by special cells designed or selected to produce high amounts of the desired protein. The produced protein may be secreted by the cells into the fluid surrounding the cells. After the protein has been produced during the fermentation it is in general purified in subsequent steps before the protein is at the intended form and purity. During the purification process the produced protein is in general separated from one or more components of the production medium, and involves generally a separation of soluble proteins from solid cell material.

If the protein is produced in sufficiently high amounts it may precipitate in crystalline form, which poses an additional challenge in the purification process that usually follows the fermentation, since the protein needs to be soluble in order to be separated from the solid cell material and/or other solid components of the production mixture. In such a situation the production mixture may be diluted with additional water or other fluids that can dissolve the precipitated protein. However even though diluting the production mixture may solve the problem of precipitated protein in the production mixture it is a less desired solution since it also means that the volume increases and consequently must the subsequent purification equipment be capable of handling a higher volume due to the dilution which generally means that larger investments and higher operational spends are necessary to cope with the increased volume.

There is therefore a need for a method for resolubilization of protein products for separation processes where the resolubilization takes place without a high increase in volume.

### SUMMARY OF THE INVENTION

In a first aspect the invention relates to a method for purifying a protein product, wherein at least part of the protein has 2-6 histidine residues located on the surface of the protein; in a process comprising the steps of:
a. Providing a fermentation broth,
b. optionally adjusting the pH to a value below the pKa of the histidine side chain;
c. Optionally holding the mixture for a period; and
d. Separating the dissolved protein product from at least part of the solid materials from the fermentation broth.

In a second aspect the invention relates to a recombinant microorganism comprising at least one polynucleotide encoding an protein of interest operably linked to one or more control sequences that direct the production of the protein of interest and at least one polynucleotide encoding a modified protein, which in comparison with the protein of interest is modified to contain 2-6 histidine residues located on the surface of the protein, the modified gene operably linked to one or more control sequences that direct the production of the modified protein.

In a third aspect the invention relates to a recombinant microorganism comprising at least one polynucleotide encoding an protein of interest operably linked to one or more control sequences that direct the production of the protein of interest and at least one polynucleotide encoding a modified protein, which in comparison with the protein of interest is modified to contain 2-6 histidine residues located on the surface of the protein, the modified gene operably linked to one or more control sequences that direct the production of the modified protein.

In a further aspect the invention related to the use of the recombinant microorganism of the second aspect to produce a protein product comprising a protein of interest and a modified protein, which in comparison with the protein of interest has the same amino acid sequence extended C- and/or N-terminally with 2-6 histidine residues.

Preferably the protein of interest is an enzyme

### SHORT DESCRIPTION OF THE FIGURES

FIG. 1 A shows SDS-page gel showing supernatants from of a lysozyme fermentation with samples taken during fermentation. In the FIGURE lane 1 is a marker, lanes 2-6 are supernatant samples of fermentation broth from a lysozyme fermentation after 97 hours, 120 hours, 144 hours, 169 hours and 192 hours respectively; and lane 7 is a purified lysozyme standard. It can be seen that the amount of lysozyme after 169 and 192 decreases compared with after 144 hours due to precipitation.

FIG. 1 B shows SDS-page gel showing supernatants from of a lysozyme fermentation with samples taken during fermentation. In the FIGURE lane 1 is a marker, lanes 2-6 are supernatant samples of fermentation broth from a lysozyme fermentation after 97 hours, 120 hours, 144 hours, 169 hours and 192 hours respectively; and lane 7 is a purified lysozyme standard. It can be seen that the amount of lysozyme increases during the whole fermentation process.

### DETAILED DESCRIPTION OF THE INVENTION

### Definitions

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Many protein products are today prepared in a fermentation process where a microorganism is fermented in a fermenter using a particular substrate and fermentation protocol. This is well known and many fermentation protocols have been described in the art. During the fermentation the microorganisms produce the intended protein and excrete the protein into the fermentation broth. After the fermentation process the liquid part, included the dissolved intended protein, is separated from the solids, such as cells, cell debris and solid remains from the substrate and the protein product may be further purified from the liquid part using a techniques known in the art. In many industrial fermentations it is however experienced that the intended protein product is produced in so high amounts that the protein precipitates forming crystalline or amorphous solids, which generates a problem in purification because it is not readily separated from the solid part.

The proteins usable in the method of the invention are in principle any proteins having a higher solubility at a pH value below the pKa of the histidine side chain, compared with the solubility at a pH value above the pKa of the histidine side chain.

Preferably the pH value below the pKa of the histidine side chain is at least 0.1 pH unit below the pKa of the histidine side chain, preferably at least 0.2 pH unit below, preferably at least 0.3 pH unit below, preferably at least 0.4 pH unit below, preferably at least 0.5 pH unit below, preferably at least 0.6 pH unit below, preferably at least 0.7 pH unit below, preferably at least 0.8 pH unit below, preferably at least 0.9 pH unit below, preferably at least 1.0 pH unit below, preferably at least 1.5 pH unit below, preferably at least 2.0 pH unit below the pKa value of the histidine side chain.

The pH value above the pKa of the histidine side chain is at least 0.1 pH unit above the pKa of the histidine side chain, preferably at least 0.2 pH unit above, preferably at least 0.3 pH unit above, preferably at least 0.4 pH unit above, preferably at least 0.5 pH unit above, preferably at least 0.6 pH unit above, preferably at least 0.7 pH unit above, preferably at least 0.8 pH unit above, preferably at least 0.9 pH unit above, preferably at least 1.0 pH unit above, preferably at least 1.5 pH unit above, preferably at least 2.0 pH unit above the pKa value of the histidine side chain.

It will be appreciated that histidine has three pKa values, one for the carboxyl group, one for the pyrrole group and one for the NH2 group. In a peptide, such as a polypeptide or a protein at least one of the carboxyl group and the NH2 group will be bound to an adjacent amino acid in a peptide bond.

The pKa of the histidine side chain is in the present specification and claim intended to mean the pKa of the imidazole ring of the histidine molecule. The pKa of the imidazole group is approximately 6.0 at 25° C. The skilled person will appreciate that the pKa will change slightly with the conditions, such as temperature, concentration and ionic strength of the solvent. For the present invention relating to purification of proteins the relevant conditions are conditions that cause relative little denaturation of the proteins, i.e. relative mild conditions. Under such conditions it can for the purpose of the present invention be assumed that the pKa of the histidine side chain is 6.0, and that is assumed in the present specification and claims unless otherwise specifically indicated.

This mean that at a pH below 6.0 the imidazole groups on the histidines, in particular the histidines exposed to the surface of a protein, will be mostly protonated and consequently positively charged, whereas at a pH above 6.0 the imidazole groups of histidines, in particular the histidines exposed to the surface of a protein, will be mostly non-protonated and consequently uncharged.

The pKa of the histidine side chain is approximately 6.0 for the free histidine. The pKa for the histidine side chain may be affected by surrounding amino acids, in particular for histidines located inside a protein structure. The histidines relevant for the present invention are histidines located on the surface of the protein of interest and are therefore in a high degree exposed to the surroundings and the change in pKa for these histidines will therefore only be small. Therefore, for the purpose of the present invention the pKa for the histidine side chain can be considered to be 6.0 independent of the surrounding amino acids.

Thus, in one embodiment the, the proteins for use in the method of the invention are proteins having a higher solubility at pH 5.5, compared with the solubility at pH 6.5; or proteins having a higher solubility at pH 5.0 compared with solubility at pH 7.0; or proteins having a higher solubility at pH 4.5 compared with solubility at pH 8.0.

The invention is based on the finding that protein having 2-6 histidines at the surface typically has a high solubility at a pH below the pKa of the histidine side chain, where the histidine side chains or a significant part thereof are positive charged, in comparison with a corresponding protein having same sequence expect for the 2-6 histidines. At a pH value above the pKa of the histidine side chain the histidine side chains are in general uncharged and typically this lead to a lower solubility at this pH.

In particular the invention relates to protein modified by inserting or substituting histidine residues in the surface regions of the protein, so the modified protein contains 2-6 histidines at the surface. The 2-6 histidines may be located internally in the primary sequence or they may be attached to the C- or N-terminus of the mature protein, or any combinations of these. Such modified proteins have the benefit of high solubility at a pH below the pKa of the histidine side chain, presumably due to the positive charges of the histidine residues at this pH; but at a pH above the pKa of the histidine side chain the modified protein will have same charge as the not modified protein and can therefore be use exactly as the unmodified protein.

The protein product may in principle be any protein prepared in a fermentation process, and the invention relates to separation processes where the protein is present in concentrations above the solubility thereof under the given conditions which accordingly leads to precipitation of some of the protein product in crystalline or amorphous form.

The protein may be a therapeutic protein or an enzyme. The enzyme may be a hydrolase, isomerase, ligase, lyase, oxidoreductase, or transferase; preferably the enzyme of interest is an alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucano-transferase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or a xylanase.

### Proteases

In a preferred embodiment the protease is a subtilisin or a metallo protease.

A subtilisin is a serine protease that uses a catalytic triad composed of Asp32, His64 and Ser221 (subtilisin BPN′ numbering).

A subtilisin may according to the peptidase classification be described as: clan SB, family S8, MEROPS ID: S08.001.

Subtilisins are described in, e.g., Barrett et al. 1998. Handbook of proteolytic enzymes. Academic press, p. 289-294. Siezen and Leunissen, Protein Science, 1997, 6&501-523 provides a description of subtilases.

There are no limitations on the origin of the protease of the invention and/or for the use according to the invention. Thus, the term protease includes not only natural or wild-type proteases, but also any mutants, variants, fragments etc. thereof exhibiting protease activity, as well as synthetic proteases, such as shuffled proteases, and consensus proteases. Such genetically engineered proteases can be prepared as is generally known in the art, e.g., by sitedirected mutagenesis, by PCR (using a PCR fragment containing the desired mutation as one of the primers in the PCR reactions), or by random mutagenesis. The preparation of consensus proteins is described in, e.g., EP 897985.

Examples of proteases for use in the present invention include wild type proteases such as the proteases having the amino acid sequences of SEQ ID NO: 2 (Savinase) or SEQ ID NO: 25 (BPN′) or variants proteases such as the Savinase variants having SEQ IDF NO: 1, SEQ ID NO: 3 or SEQ ID NO: 4. Preferred proteases for use in the present inventions are proteases having at least 80% sequence identity, e.g at least 90% sequence identity, e.g. at least 95% sequence identity, e.g. at least 96% sequence identity, e.g at least 97% sequence identity, e.g. at least 98% sequence identity, e.g. at least 99% sequence identity to one of SEQ ID NO:1, SEQ ID NO: 2, SEQ ID NO: 3, SEQ ID NO: 4 or SEQ ID NO: 25.

### Amylases

Suitable amylases (alpha and/or beta) include those of bacterial origin. Chemically modified or protein engineered mutants are included. Amylases include, for example, alphaamylases obtained from Bacillus, e.g. al strain of B. licheniformis, described in more detail in GB 1,296,839.

### Cellulases

Suitable cellulases include those of bacterial or fungal origin. Chemically modified or protein engineered mutants are included (including substitutions, insertions, and/or deletions). Suitable cellulases include cellulases from the genera Bacillus, Pseudomonas, Humicola, Fusarium, Thielavia, Acremonium, e.g. the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

### Lipases

Suitable lipases include those of bacterial or fungal origin including protein engineered mutants (including substitutions, insertions and/or deletions). Suitale lipases include lipases from the genera Humicola and Rhizomucor, e.g the fungal lipases produced from Humocola lanuginose and Rhizomucor mihei.

### Oxidoreductases

Oxidoreductases that may be treated according to the invention include peroxidases (EC 1.11.1.7), and oxidases such as laccases, and catalases (EC 1.11.1.6).

### Lysozymes

The term “lysozyme” activity is defined herein as an O-glycosyl hydrolase, which catalyses the hydrolysis of the glycosidic bond between two or more carbohydrates, or between a carbohydrate and a non-carbohydrate moiety. Lysozymes cleave the glycosidic bond between certain residues in mucopolysaccharides and mucopeptides of bacterial cell walls, such as 1,4-beta-linkages between N-acetylmuramic acid and N-acetyl-D-glucosamine residues in a peptidoglycan and between N-acetyl-D-glucosamine residues in chitodextrins, resulting in bacteriolysis. Lysozyme belongs to the enzyme class EC 3.2.1.17.

Examples of lysozymes for use in the present invention includes lysozymes disclosed in WO 2003/076253. Preferred lysozymes for use in the present inventions are lysozymes having at least 80% sequence identity, e.g at least 90% sequence identity, e.g. at least 95% sequence identity, e.g. at least 96% sequence identity, e.g at least 97% sequence identity, e.g. at least 98% sequence identity, e.g. at least 99% sequence identity to SEQ ID NO:18.

The pH in the fermentation broth at the end of the fermentation process and the low pH are selected so that the net charge of the protein of interest changes between the pH at the end of the fermentation and the low pH. One preferred way to secure this is selecting a protein of interest having 2-6 amino acids having pKa values between the pH value at the end of the fermentation and the low pH value located at the surface of the protein of interest.

A preferred example of an amino acid residue having pKa values in a suitable range taking the pH tolerance of commonly used host cells and pH stability of the protein of interest can be mentioned histidine having a pKa of the side chain of about 6.0.

The pH in the fermentation broth at the end of the fermentation may depend on several parameters such as host organism, composition of the fermentation medium, oxygen supply, extend of pH regulation during the process and in general the conditions under the fermentation process. However, typical industrial fermentation processes are pH regulated and the pH at the end of the fermentation is determined by the pH regulation applied to the particular process.

The proteins for use according to the invention may be natural proteins, understood as proteins having same amino acid sequence as a protein naturally found in nature; or it may be an engineered protein where the amino acid sequence has been altered by man with the consequence that proteins having such amino acid sequences are not found naturally in nature.

One preferred class of such proteins for use in the method of the invention are proteins having 2-6 histidine residues located on the surface of the protein. Such proteins may be natural proteins or it may be engineered, e.g. engineered to contain 2-6 histidines on the surface thereof. The 2-6 histidine restudies located on the surface may be located internally in the primary amino acid sequence of the protein or they may be located in one or the other end of the amino acid sequence of the protein, or it may even be a combination thereof.

One preferred class of engineered proteins for use in the method of the invention are proteins having a His tag attached to the N- or the C-terminal or a protein. In the present application a His-tag is intended to mean a short stretch of amino acids comprising 2-6 adjacent histidine residues. The His tag may contain a protease cleavage site that allow for removal of the his-tag after purification of the protein including the his-tag, and thereby obtain a protein devoid of any his tag residues.

Other engineered proteins for use in the method of the invention and enzymes engineered to contain 2-6 histidines internally in the primary amino acid sequence and located on the surface of the protein. Such proteins may be designed as described in the co-pending application Ser. No. 14/162,434.6 filed with the European Patent Office and named “ENZYME VARIANTS AND POLYNUCLEOTIDES ENCODING SAME (included herein by reference) and the teachings thereof also applies for the present specifications and claims.

In general the pH in a fermentation process is controlled during the process in order to obtain the optimal product yield and quality. This is well known in the art. Using proteins having 2-6 histidines located on the surface may provide for a particular benefit in that is it possible to impact the solubility of the protein of interest by controlling the pH. Thus the solubility can be increased be lowering the pH to a pH value below the pKa of the histidine side chain, and the solubility can be decreased by raising the pH to a pH value above the pKa of the histidine side chain.

This has a particular benefit for fermentations providing both an intended product that is susceptible for protease degradation and in addition providing a protease ending up in the fermentation broth. In such a situation is may be beneficial to perform the process under conditions where the protein of interest precipitated during the fermentation, because proteins in general are less susceptible to protease degradation in solid state, and the dissolve the protein during purification in order to separate the intended protein from the solid parts.

It is known that many microorganism produces proteases during fermentation, either as the intended product, as a side activity or as result of lysis of some of the cells, which all may lead to some degradation of the intended protein of interest and thereby loss of product or reduction of product quality. In particular in fermentation for production of proteases, the produced protease will degrade the protein present, known as autoproteolysis, and therefore it may be beneficial to perform a protease fermentation process under conditions where the protease precipitates during fermentation, and is thereby protected against autoproteolysis, and subsequent the protein is resolubilized during purification where the product is separated from the solids using suitable separation technology.

This may according to the invention be done by fermenting at a pH above 6.0 and the lower the pH to a pH below 6.0 during at least part of the purification. The fermentation may for example be performed at a pH above 6.0, e.g. above 6.2; e.g. above 6.5 e.g. above 7.0 and the purification may at least in part be performed at a pH below 5.8, e.g. below 5.5, e.g. below 5.0.

In one preferred embodiment the protein product is produced by an engineered microorganism engineered to contain one or more genes encoding a gene of interest and one or more genes encoding a modified version of the gene of interest, modified so that the encoded protein contain 2-6 histidines internally in the primary amino acid sequence and located on the surface of the protein, or a His-tag of 2-6 histidines attached to the N- and/or the C-terminal of the protein; and wherein the gene(s) of interest and the modified gene(s) of interest are all expressed during fermentation of the microorganism. It has surprisingly been found that the protein of interest produced by such an engineered microorganism has a higher solubility that the corresponding microorganism without the modified gene of interest and also that the precipitated protein of interest is readily soluble by shifting the pH below 6.0.

The copy number of the gene of interest and the modified gene of interest in the engineered microorganism may be in the range of 1-20, such as 1-10, such as 1-5. The copy number of the gene of interest may or may not be the same as the copy number of the modified gene of interest. In one preferred embodiment the copy number of the modified gene of interest is 1 and the copy number of the gene of interest is 1, 2, 3, 4, 5, 6, 7 or 8, in another preferred embodiment the copy number of the modified gene of interest is 2 and the copy number of the gene of interest is 1, 2, 3, 4, 5, 6, 7 or 8.

### Polynucleotides

The present invention also relates to isolated polynucleotides encoding a polypeptide, as described herein.

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dana (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase Ill, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. Other examples of regulatory sequences are those that allow for gene amplification. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis daI genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocaffimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Fermentation Broth

The fermentation broth according to the invention comprises the cells producing the protein of interest, and the protein of interest partly present as crystals and/or amorphous precipitate.

Any cell known in the art may be used. The cell may be a microorganism or a mammalian cell. The microorganism according to the invention may be a microorganism of any genus.

In a preferred embodiment, the protein of interest may be obtained from a bacterial or a fungal source.

For example, the protein of interest may be obtained from a gram positive bacterium such as a Bacillus strain, e.g., Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis; or a Streptomyces strain, e.g., Streptomyces lividans or Streptomyces murinus; or from a gram negative bacterium, e.g., E. coli or Pseudomonas sp. In a preferred embodiment the cell is a Bacillus cell.

The protein of interest may be obtained from a fungal source, e.g. from a yeast strain such as a Candida, Kluyveromyces, Pichia, Saccharomyces, Schizosaccharomyces, or Yarrowia strain, e.g., Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis strain.

The protein of interest may be obtained from a filamentous fungal strain such as an Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma strain, in particular the polypeptide of interest may be obtained from an Aspergillus aculeatus, Aspergillus awamori, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenaturn, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride strain.

Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

In a preferred embodiment the cells of the invention are single cells. Some fungi may be produced in a yeast-like form. The fungi cells may also be fragmented and/or disrupted as described in WO 2005/042758.

For purposes of the present invention, the term “obtained from” as used herein in connection with a given source shall mean that the protein of interest is produced by the source or by a cell in which a gene from the source has been inserted.

The cells may be fermented by any method known in the art. The fermentation medium may be a complex medium comprising complex nitrogen and/or carbon sources, such as soybean meal, cotton seed meal, corn steep liquor, yeast extract, casein hydrolysate, molasses, and the like. The fermentation medium may be a chemically defined media, e.g. as defined in WO 98/37179.

Several commercial ingredients in fermentation media are not soluble and contains indigestible components that will remain in the fermentation medium during the fermentation and which need to be separated from the desired protein after the fermentation. The skilled person will therefore appreciate that the fermentation medium after the fermentation will according to the invention comprise several solid components including the protein of interest in partially crystalline or amorphous form, cells and cell debris and insoluble remains of the fermentation medium.

The fermentation may be performed as a fed-batch, a repeated fed-batch or a continuous fermentation process.

During the fermentation the protein of interest is produced and for efficient industrial fermentations, it is frequently observed that the protein of interest precipitates because it is produced in concentrations above the solubility of the protein of interest.

Precipitation of the protein of interest provides a challenge for the skilled person during the purification of the protein of interest after the fermentation where cells, cell debris and solid remains of the growth medium is separated from the fluid by known methods for solid/fluid separations such as filtration. If the protein of interest is completely or partially available in solid form, it will follow the solid in this separation and thereby reducing the yield.

The method of the invention provides a method for purifying a protein of interest where the protein of interest is present in solid form, either in crystalline form or in amorphous form or a mixture thereof. It is well known in the art that proteins as well as other chemical compounds, precipitates from a solution when the concentration of the protein exceeds the limit for solubility. Thus the method of the invention is particular useful when producing a protein or interest in high amounts. Thus preferably the concentration of the protein of interest in the fermentation broth is preferably higher than 3 g/l, such as higher than 4 g/l, such as higher than 5 g/l, such as higher than 6 g/l, such as higher than 7 g/l, such as higher than 8 g/l, such as higher than 9 g/l, such as higher than 10 g/l, such as higher than 11 g/l, such as higher than 12 g/l, such as higher than 13 g/l, such as higher than 14 g/l, such as higher than 15 g/l, such as higher than 16 g/l, such as higher than 17 g/l, such as higher than 18 g/l, such as higher than 19 g/l, such as higher than 20 g/l.

The term solid form is in the present description and claims used to describe the solid form found in the fermentation broth when the production of the protein of interest has reached a sufficiently high level that exceed the solubility limit of the particular protein. The solid form may by in crystalline form meaning that the molecules are arranged regularly in a structure that is characterized by regular shapes and angels, and with same organization of the molecules throughout the whole structure. Typically crystals can diffract light in fixed angels due to the regular organization of the crystals. The solid form may also be in amorphous form which is understood as a less regular structure where the molecules are arranged less regular than found in crystals and the organization of the molecules differs from one part of the structure to other parts of the structure. The solid form may also be in a partially crystalline form where part of the material is in crystalline form intermixed with other parts of the solid material being in amorphous form. The solid form wherein the protein of interest exist in in the fermentation broth is not in any way limiting for the invention, in the contrary the method of the invention is suitable for any protein of interest having the property of being more soluble at low pH compared with the solubility at the pH of the fermentation broth.

For adjustment of pH virtually any acid can be used. The acid may be inorganic or organic. Some examples are hydrochloric acid, sulphuric acid, sulphurous acid, nitrous acid, phosphoric acid, acetic acid, citric acid, and formic acid. The skilled person will be capable of selecting a suitable acid for the purpose of the invention in general based on cost and consideration regarding which acids would be acceptable in the following purification process. Preferred acids are phosphoric acid, formic acid, citric acid, and acetic acid.

When the pH of the fermentation broth is adjusted to the low pH value, the protein of interest will start to resolubilize because the solubility of the protein has increased due to the change in pH. The dissolution of the protein of interest in solid form may be quick or it may be slow depending of the particular conditions in the container and the properties of the particular protein. Like other dissolution processes it will be accelerated in the mixture is agitated e.g. by stirring the mixture compared to the corresponding situation without agitation of the mixture.

After the pH adjustment a holding period may be applied in order to allow the protein of interest to dissolve before the fermentation broth is treated in the post treatment process, e.g. in one or further purification steps. The holding period should be of a sufficient length to ensure a satisfactory dissolution of the protein of interest before post-treatment. Typically, the holding period will be at least 5 minutes, e.g. at least 10 minutes, e.g. at least 20 minutes, e.g. at least 30 minutes, e.g. at least 40 minutes, e.g. at least 50 minutes, e.g. at least 60 minutes, e.g. at least 70 minutes, e.g. at least 80 minutes, e.g. at least 90 minutes, e.g. at least 100 minutes, e.g. at least 110 minutes, e.g. at least 120 minutes.

After the pH adjustment and optional holding period the fermentation broth with the protein of interest is post-treated in order to achieve the final desired product. Typically the first step of the post-treatment is a separation process where the liquid part of the fermentation broth containing the protein of interest in solution is separated from insoluble parts, such as cells and cell debris and remains of the growth medium. The invention is not limited to any particular type of separation process but any type of separation process capable of separating a fluid from insolubles can in principle be used, such as filtration, centrifugation or decantation. After the separation further steps may be applied in order to achieve the protein of interest in the desired form, purity and formulation, such as concentration, chromatography, stabilization, spray drying, granulation.

The method of the invention may further comprise a pretreatment of the fermentation broth before the solid/liquid separation, such as a dilution step, where the fermentation broth is diluted with water or an aqueous solution, addition of salts or addition of other compounds having a beneficial effect during purification, such as polymers or stabilizers etc. The pretreatment step may take place before or after adjusting the pH to a value below the pKa of histidine.

### Preferred Embodiments

The invention is now described by the following embodiments:
1. A method for purifying a protein product, wherein at least part of the protein has 2-6 histidine residues located on the surface of the protein; in a process comprising the steps of:
   a. Providing a fermentation broth,
   b. optionally adjusting the pH to a value below the pKa of the histidine side chain;
   c. Optionally holding the mixture for a period; and
   d. Separating the dissolved protein product from at least part of the solid materials from the fermentation broth.
2. The method of embodiment 1, wherein the pKa of the histidine side chain is 6.0.
3. The method of embodiment 1 or 2, wherein the 2-6 histidines located on the surface of the protein are located internally in the primary sequence.
4. The method of embodiment 3 where at least one of the 2-6 histidine residues at the surface of the protein of interest is provided by substitution or insertion.
5. The method of embodiment 1 or 2, wherein the 2-6 histidines located on the surface of the protein are in form of a C- and/or N-terminal extension of the protein.
6. The method of embodiment 1-5, wherein the protein product is an enzyme.
7. The method of embodiment 6, wherein the enzyme is selected among hydrolase, isomerase, ligase, lyase, oxidoreductase, or transferase.
8. The method of embodiment 7, wherein the enzyme is an alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucano-transferase, glucoamylase, invertase, laccase, lipase, lysozyme, mannosidase, mutanase, oxidase, pectinolytic enzyme, peroxidase, phytase, polyphenoloxidase, proteolytic enzyme, ribonuclease, transglutaminase, or a xylanase.
9. The method of embodiment 8, wherein the enzyme is a protease, selected among metalloproteases and subtilases.
10. The method of embodiment 9, wherein the enzyme is a protease having at least 80% sequence identity to SEQ ID NO: 1, SEQ ID NO: 2, SEQ ID NO: 3, SEQ ID NO: 4 or SEQ ID NO: 25.
11. The method of embodiment 10, wherein the enzyme is a protease having at least 85% sequence identity to SEQ ID NO: 1, SEQ ID NO: 2, SEQ ID NO: 3, SEQ ID NO: 4 or SEQ ID NO: 25.
12. The method of embodiment 11, wherein the enzyme is a protease having at least 90% sequence identity to SEQ ID NO: 1, SEQ ID NO: 2, SEQ ID NO: 3, SEQ ID NO: 4 or SEQ ID NO: 25.
13. The method of embodiment 12, wherein the enzyme is a protease having at least 95% sequence identity to SEQ ID NO: 1, SEQ ID NO: 2, SEQ ID NO: 3, SEQ ID NO: 4 or SEQ ID NO: 25.
14. The method of embodiment 13, wherein the enzyme is a protease having at least 96% sequence identity to SEQ ID NO: 1, SEQ ID NO: 2, SEQ ID NO: 3, SEQ ID NO: 4 or SEQ ID NO: 25.
15. The method of embodiment 14, wherein the enzyme is a protease having at least 97% sequence identity to SEQ ID NO: 1, SEQ ID NO: 2, SEQ ID NO: 3, SEQ ID NO: 4 or SEQ ID NO: 25.
16. The method of embodiment 15, wherein the enzyme is a protease having at least 98% sequence identity to SEQ ID NO: 1, SEQ ID NO: 2, SEQ ID NO: 3, SEQ ID NO: 4 or SEQ ID NO: 25.
17. The method of embodiment 16, wherein the enzyme is a protease having at least 99% sequence identity to SEQ ID NO: 1, SEQ ID NO: 2, SEQ ID NO: 3, SEQ ID NO: 4 or SEQ ID NO: 25.
18. The method of embodiment 8, wherein the enzyme is a lysozyme having at least 80% sequence identity to SEQ ID NO: 18.
19. The method of embodiment 18, wherein the enzyme is a lysozyme having at least 85% sequence identity to SEQ ID NO: 18.
20. The method of embodiment 19, wherein the enzyme is a lysozyme having at least 90% sequence identity to SEQ ID NO: 18.
21. The method of embodiment 20, wherein the enzyme is a lysozyme having at least 95% sequence identity to SEQ ID NO: 18.
22. The method of embodiment 21, wherein the enzyme is a lysozyme having at least 96% sequence identity to SEQ ID NO: 18.
23. The method of embodiment 22, wherein the enzyme is a lysozyme having at least 97% sequence identity to SEQ ID NO: 18.
24. The method of embodiment 23, wherein the enzyme is a lysozyme having at least 98% sequence identity to SEQ ID NO: 18.
25. The method of embodiment 24, wherein the enzyme is a lysozyme having at least 99% sequence identity to SEQ ID NO: 18.
26. The method of any of the preceding embodiments, wherein the solubility of the protein of interest at pH 4.5 is at least 10% higher than the solubility at pH 7.0, preferably at least 20% higher, preferably at least 30% higher, preferably at least 40% higher, preferably at least 50% higher, preferably at least 60% higher, preferably at least 70% higher, preferably at least 80% higher, preferably at least 90% higher, preferably at least 100% higher.
27. The method according to any of the preceding embodiments, wherein the concentration of the protein product in the fermentation broth is at least 3 g/l, such as at least 4 g/l, such as at least 5 g/l, such as at least 6 g/l; such as at least 7 g/l, such as at least 8 g/l, such as at least 9 g/l; such as at least 10 g/l, such as at least 11 g/l, such as at least 12 g/l; such as at least 13 g/l, such as at least 14 g/l, such as at least 15 g/l; such as at least 16 g/l, such as at least 17 g/l, such as at least 18 g/l; such as at least 19 g/l, such as at least 20 g/l.
28. The method according to any of the preceding embodiments, wherein the pH in step b) is adjusted to a pH value below, 6.0, preferably below 5.5, preferably below 5.0, preferably below 4.5.
29. The method according to any of the preceding embodiments, comprising a holding period in step c and wherein the holding period is in the range of 10 seconds to 90 minutes, preferably in the range of 1 minutes to 90 minutes, preferably in the range of 1 minute to 60 minutes, preferably in the range of 1 minutes to 30 minutes, such as in the range of 5 minutes to 30 minutes, and most preferred in the range of 10 to 20 minutes.
30. The method of any of the preceding embodiments, wherein the fermentation broth is provided by cultivating a microorganism in a growth medium.
31. The method of embodiment 30, wherein the microorganism is selected among bacteria and fungi.
32. The method of embodiment 31, wherein the microorganism is selected among bacteria, belonging to the genus Bacillus, such as Bacillus subtilis, B. lentus and B. lichiniformis.
33. The method of embodiment 31, wherein the microorganism is selected among fungi belonging to the generi Aspergillus, Trichoderma, Penicillum, Fusarium, such as A. niger, A. awamori, A. oryzae, A. sojae, T. reesei, T. longibrachiatum or T. viride; or yeasts preferably belonging to the generi Saccharomyces, Pichia, Candida, Hanensula, Klyveromyces; such as S. cerevisiae, S. ovarum, P. Pastoris, K. lactis.
34. The method of any of the preceding embodiments, wherein the separation in step d is performed using filtration, centrifugation or decantation.
35. The method according to any of the preceding embodiments wherein the method further comprises a pre-treatment step before the separation in step d.
36. The method of embodiment 35, wherein the pre-treatment step is selected among a dilution, salt addition and addition of a polymer.
37. The method of any of the preceding embodiments, wherein the protein product contains a protein of interest having a given amino acid sequence and a modified protein having same amino acid sequence except for a C- and/or N-terminal extension of 2-6 histidine residues.
38. The method of embodiment 37, wherein the fermentation broth is provided by fermenting a substrate with a recombinant microorganism comprising one or more copies of a gene encoding the protein of interest, and one or more copies of a modified gene encoding the modified protein consisting of the sequence of the protein of interest extended C- and/or N-terminally with 2-6 histidine residues.
39. The method of embodiment 38, wherein the recombinant microorganism contains two, three, four, five, six, seven or eight copies of the gene encoding the protein of interest, and one or two copies of the modified gene.
40. The method of any of the preceding embodiments comprising a holding period in step c and wherein the holding period is in the range of 10 seconds to 90 minutes, preferably in the range of 1 minutes to 90 minutes, preferably in the range of 1 minute to 60 minutes, preferably in the range of 1 minutes to 30 minutes, such as in the range of 5 minutes to 30 minutes, and most preferred in the range of 10 to 20 minutes.
41. A recombinant microorganism comprising at least one polynucleotide encoding an protein of interest operably linked to one or more control sequences that direct the production of the protein of interest and at least one polynucleotide encoding a modified protein, which in comparison with the protein of interest is modified to contain 2-6 histidine residues located on the surface of the protein, the modified gene operably linked to one or more control sequences that direct the production of the modified protein.
42. The recombinant microorganism of embodiment 41, which is a prokaryotic cell, preferably a Gram-positive cell, more preferably a Bacillus cell; most preferably a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis or Bacillus thuringiensis cell.
43. The recombinant microorganism of embodiment 41, which is an eukaryotic cell, preferably a fungal cell, more preferably an Aspergillus, Trichoderma or Saccharomyces or Pichia cell; most preferably an Aspergillus niger, Aspergillus oryzae, Aspergillus awamori, Aspergillus aculeatus, Trichoderma reesei, Trichoderma harzianum Trichoderma virede, Saccharomyces cerevisiae, Saccharomyces ovarum or Pichia pastoris cell.
44. The recombinant microorganism of any of embodiments 41-43, which comprises at least two copies of the polynucleotide encoding the protein of interest, preferably at least three copies, more preferably at least four copies and most preferably at least five copies of the polynucleotide encoding the protein of interest.
45. The recombinant microorganism of any of embodiments 41-44 which comprises at least two different polynucleotides encoding the same protein of interest, preferably at least three, more preferably at least four and most preferably at least five polynucleotides encoding the same protein of interest.
46. The recombinant microorganism according to any of the embodiments 41 to 45, wherein the protein of interest is an enzyme.
47. The recombinant microorganism of embodiment 46, wherein the enzyme is a hydrolase, isomerase, ligase, lyase, oxidoreductase, or transferase.
48. The recombinant miocroorganism of embodiment 47, wherein the enzyme is an alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucano-transferase, glucoamylase, invertase, laccase, lipase, lysozyme, mannosidase, mutanase, oxidase, pectinolytic enzyme, peroxidase, phytase, polyphenoloxidase, proteolytic enzyme, ribonuclease, transglutaminase, or a xylanase.
49. The recombinant microorganism of any of embodiment 48, wherein the enzyme is a protease; preferably the protease is a metalloprotease or an subtilase.
50. The recombinant microorganism of embodiment 49, wherein the protease has an amino acid sequence with at least 80% sequence identity, preferably at least 90% sequence identity, preferably at least 95% sequence identity, preferably at least 96% sequence identity, preferably at least 97% sequence identity preferably at least 98% sequence identity, preferably at least 99% sequence identity to one of SEQ ID NO: 1, SEQ ID NO: 2, SEQ ID NO: 3, SEQ ID NO: 4 or SEQ ID NO: 25.
51. The recombinant microorganism of embodiment 50, wherein the protease is selected among SEQ ID NO: 1, SEQ ID NO: 2, SEQ ID NO: 3, SEQ ID NO: 4 or SEQ ID NO: 25.
52. The recombinant microorganism of embodiment 48, wherein the enzyme is a lysozyme; preferably the lysozyme is a GH25 lysozyme.
53. The recombinant microorganism of embodiment 52, wherein the lysozyme has an amino acid sequence with at least 80% sequence identity, preferably at least 90% sequence identity, preferably at least 95% sequence identity, preferably at least 96% sequence identity, preferably at least 97% sequence identity preferably at least 98% sequence identity, preferably at least 99% sequence identity to SEQ ID NO: 18.
54. The recombinant microorganism of embodiment 53, wherein the lysozyme has the amino acid sequence of SEQ ID NO: 18.
55. The recombinant microorganism of any of embodiments 41-54, wherein the polynucleotides are integrated into the chromosome of the host cell in different loci.
56. A method of producing an enzyme, said method comprising at step of cultivating a cell as defined in any of embodiments 41-54 under conditions conducive for production of the enzyme.
57. The method of embodiment 56, further comprising a step of recovering the enzyme.
58. A recombinant microorganism comprising at least one polynucleotide encoding an protein of interest operably linked to one or more control sequences that direct the production of the protein of interest and at least one polynucleotide encoding a modified protein, which in comparison with the protein of interest is modified to contain 2-6 histidine residues located on the surface of the protein, the modified gene operably linked to one or more control sequences that direct the production of the modified protein.
59. The protein product of embodiment 58, wherein the modified protein comprises 2-6 histidine residues attached to the C- and/or N-terminus of the protein.
60. The protein product of embodiment 58 or 59, wherein the protein of interest is an enzyme.
61. The protein product of embodiment 60, wherein the enzyme is a hydrolase, isomerase, ligase, lyase, oxidoreductase, or transferase.
62. The protein product of embodiment 61, wherein the enzyme is an alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucano-transferase, glucoamylase, invertase, laccase, lipase, lysozyme, mannosidase, mutanase, oxidase, pectinolytic enzyme, peroxidase, phytase, polyphenoloxidase, proteolytic enzyme, ribonuclease, transglutaminase, or a xylanase.
63. The protein product of embodiment 62, wherein the enzyme is a protease; preferably the protease is a metalloprotease or an subtilase.
64. The protein product of embodiment 63, wherein the protease has an amino acid sequence with at least 80% sequence identity, preferably at least 90% sequence identity, preferably at least 95% sequence identity, preferably at least 96% sequence identity, preferably at least 97% sequence identity preferably at least 98% sequence identity, preferably at least 99% sequence identity to one of SEQ ID NO: 1, SEQ ID NO: 2, SEQ ID NO: 3, SEQ ID NO: 4 or SEQ ID NO: 25.
65. The protein product of embodiment 64, wherein the protease is selected among SEQ ID NO: 1, SEQ ID NO: 2, SEQ ID NO: 3, SEQ ID NO: 4 or SEQ ID NO: 25.
66. The protein product of embodiment 62, wherein the enzyme of interest is a lysozyme; preferably the lysozyme is a GH25 lysozyme.
67. The protein product of embodiment 66, wherein the protease has an amino acid sequence with at least 80% sequence identity, preferably at least 90% sequence identity, preferably at least 95% sequence identity, preferably at least 96% sequence identity, preferably at least 97% sequence identity preferably at least 98% sequence identity, preferably at least 99% sequence identity to SEQ ID NO: 18.
68. The protein product of embodiment 67, wherein the lysozyme has the amino acid sequence of SEQ ID NO: 18.

The invention is now further described with examples that are provided for illustration only and should not be considered limiting in any ways.

### Examples

### Materials and Methods

### Protease Assay (Suc-AAPF-pNA Assay)

pNA substrate: Suc-AAPF-pNA (Bachem L-1400).
Temperature: Room temperature (25° C.)
Assay buffer: 100 mM succinic acid, 100 mM HEPES, 100 mM CHES, 100 mM CABS, 1 mM CaCl2, 150 mM KCl, 0.01% Triton X-100, pH 9.0.
   20 μl protease (diluted in 0.01% Triton X-100) was mixed with 100 μl assay buffer. The assay was started by adding 100 μl pNA substrate (50 mg dissolved in 1.0 ml DMSO and further diluted 45× with 0.01% Triton X-100). The increase in OD405 was monitored as a measure of the protease activity.

### Determination of Lysozyme Activity (LSU(A)DV)

Lysozyme (EC 3.2.1.17) is an enzyme that degrades the peptidoglycan in Gram positive bacteria cell walls. In the analysis of Lysozyme, Micrococcus lysodeikticus ATCC no. 4698 (Sigma M3770) is degraded, whereby the absorbance at 450 nm is decreased, measured under the conditions in table 1. The decrease in absorbance is proportional to the LSU(A)DV enzyme activity present in the sample.

TABLE 1

| Parameter | Reaction conditions | |
|---|---|---|
| Temperature | 37° | C. |
| pH | 4.5 | |
| Buffer | 0.1M Acetate, 50 mM | |
| | NaCl, 0.1% Triton X-100 | |
| Substrate conc. | 0.218 | g/L |
| Enzyme conc. | 3.833-11.50 LSU(A)DV/ml | |
| (conc. of low/high standard in | | |
| reaction mixture) | | |
| Reaction time | 15 | min. |
| Wave length | 450 | nm |

### Standard (193-81114: 23000 LSU(A)DV/g)

0.5000 g/100 ml Acetate 0.1 M, NaCl 50 mM, Triton-X100 0.1%, pH 4.5

### General Molecular Biological Methods

General methods of PCR, cloning, ligation nucleotides etc. are well-known to a person skilled in the art and may for example be found in in “Molecular cloning: A laboratory manual”, Sambrook et al. (1989), Cold Spring Harbor lab., Cold Spring Harbor, N.Y.; Ausubel, F. M. et al. (eds.); “Current protocols in Molecular Biology”, John Wiley and Sons, (1995); Harwood, C. R., and Cutting, S. M. (eds.); “DNA Cloning: A Practical Approach, Volumes I and II”, D. N. Glover ed. (1985); “Oligonucleotide Synthesis”, M. J. Gait ed. (1984); “Nucleic Acid Hybridization”, B. D. Hames & S. J. Higgins eds (1985); “A Practical Guide To Molecular Cloning”, B. Perbal, (1984).

TABLE 2
General PCR conditions:

| Component | Volume | Final Concentration | |
|---|---|---|---|
| 10x Buffer for KOD -Plus- | 5 μl | 1x | |
| (Toyobo) | | | |
| 2 mM dNTPs | 5 μl | 0.2 | mM each |
| 25 mM MgSO₄ | 2 μl | 1.0 | mM |
| 10 pmol/μl Primer #1 | 1.5 μl | 0.3 | μM |
| 10 pmol/μl Primer #2 | 1.5 μl | 0.3 | μM |

| | | |
|---|---|---|
| Template DNA | X μl | |
| Plasmid DNA | 1-50 ng/50 μl | |
| PCR grade water | Y μl | |
| KOD-Plus-(1.0 U/μl) | 1 μl | 1.0 U/50 μl |
| Total reaction volume | 50 μl | |

### 3-Step Cycle:

| | | |
|---|---|---|
| Pre-denaturation: 94° C., 2 min. | | |
| Denaturation: 94° C., 15 sec. | | |
| Annealing: Tm-[5-10] ° C.*, 30 sec. | {close oversize brace} | 35 cycles |
| Extension: 68° C., 1 min./kb | | |

### Media and Reagents

Chemicals used for buffers and substrates were commercial products of analytical grade:
Cove: 342.3 g/L Sucrose, 20 ml/L COVE salt solution, 10 mM Acetamide, 30 g/L noble agar.
Cove top agar: 342.3 g/L Sucrose, 20 ml/L COVE salt solution, 10 mM Acetamide, 10 g/L low melt agarose
Cove-2: 30 g/L Sucrose, 20 ml/L COVE salt solution, 10 mM Acetamide, 30 g/L noble agar.
COVE salt solution is composed of 26 g KCl, 26 g MgSO₄.7H₂O, 76 g KH₂PO₄ and 50 ml Cove trace metals, water to 1 litre.
Trace metal solution for COVE is composed of 0.04 g NaB₄O₇.10H₂O, 0.4 g of CuSO₄.5H₂O, 1.2 g of FeSO₄.7H₂O, 1.0 g of MnSO₄.H2O, 0.8 g of Neutral amylase II MoO₂.2H₂O, and 10.0 g of ZnSO₄.7H₂O, water to 1 litre.
Amyloglycosidase trace metal solution is composed of 6.8 g ZnCl₂.7H₂O, 2.5 g CuSO₄.5H₂O, 0.24 g NiCl₂.6H₂O, 13.9 g FeSO₄.7H₂O, 13.5 g MnSO₄—H₂O and 3 g citric acid, water to 1 litre.
YPG is composed of 4 g of yeast extract, 1 g of KH₂PO₄, 0.5 g of MgSO₄.7H₂O and 15 g of Glucose (pH 6.0), water to 1 litre.
STC buffer is composed of 0.8 M of sorbitol, 25 mM of Tris (pH 8), and 25 mM of CaCl₂, water to 1 litre.
   STPC buffer is composed of 40% PEG4000 in STC buffer.

### Aspergillus Niger Transformation

Aspergillus transformation was done as described by Christensen et al.; Biotechnology 1988 6 1419-1422. The preferred procedure is described below.

The Aspergillus niger host strain was inoculated to 100 ml of YPG medium supplemented with 10 mM uridine and incubated for 16 hrs at 32° C. at 80 rpm. Pellets were collected and washed with 0.6 M KCl, and resuspended 20 ml 0.6 M KCl containing a commercial β-glucanase product (GLUCANEX™, Novozymes A/S, Bagsværd, Denmark) at a final concentration of 20 mg per ml. The suspension was incubated at 32° C. at 80 rpm until protoplasts were formed, and then washed twice with STC buffer. The protoplasts were counted with a hematometer and resuspended and adjusted in an 8:2:0.1 solution of STC:STPC:DMSO to a final concentration of 2.0×10⁷ protoplasts/ml. Approximately 4 μg of plasmid DNA was added to 100 μl of the protoplast suspension, mixed gently, and incubated on ice for 30 minutes. One ml of SPTC was added and the protoplast suspension was incubated for 20 minutes at 37° C. After the addition of 10 ml of 50° C. Cove top agarose, the reaction was poured onto Cove agar plates and the plates were incubated at 32° C. for 5 days.

### SDS-PAGE

SDS gel used for lysozyme analysis was Any kD™ Mini-PROTEAN® TGX Stain-Free™ gels from BioRad. Sixteen ul of samples was loaded on the gel (Eight μl of each sample was mixed with 8 ul of loading buffer). Ten ul of MW Marker: (Low Molecular Weight Calibration Kit for SDS Electrophoresis #17-0446-01 from Amersham) was also applied. The gel was electrophoresed at a constant voltage of 200V for 25 min in 1×SDS buffer (BioRad) and analysed by using the BioRad criterion system as recommend by the manufacturer.

### Example 1

### Preparation and Expression of Variants

The following summarizes the mutation and introduction of an expression cassette into Bacillus subtilis. All DNA manipulations were done by PCR (e.g. Sambrook et al.; Molecular Cloning; Cold Spring Harbor Laboratory Press) and can be repeated by everybody skilled in the art.

Recombinant B. subtilis constructs encoding subtilase variants were used to inoculate shakeflasks containing a rich media (e.g. PS-1: 100 g/L Sucrose (Danisco cat.no. 109-0429), 40 g/L crust soy (soy bean flour), 10 g/L Na₂HPO₄.12H₂O (Merck cat.no. 6579), 0.1 ml/L replaceDowfax63N10 (Dow). Cultivation typically takes 4 days at 30° C. shaking with 220 rpm.

Following proteins were generated

### Reference: SEQ ID NO:1

1HIS: SEQ ID NO: 1 with 1 Histidine attached to N-terminus
2HIS: SEQ ID NO: 1 with 2 Histidine attached to N-terminus
3HIS: SEQ ID NO: 1 with 3 Histidine attached to N-terminus
4HIS: SEQ ID NO: 1 with 4 Histidine attached to N-terminus
3 intHIS: SEQ ID NO: 1+V239H+N243H+N247H

### Example 2

The reference and variants generated in Example 1 were fermented in standard lab scale fermentors using the method described in EP 1 520 012 B1, Example 2, without addition of MGP.

It was observed that the variants precipitated during the fermentation. The activities in the fermentations showed that the fermentations of Reference, 1HIS, 2HIS, 3HIS and 4HIS gave approximately same yield, whereas the 3intHIS variant fermentation gave a lower yield that the reference.

### Example 3

The fermentation broths from Example 2 were diluted three fold with water and pH were adjusted to pH 4.5 at 40° C. using HCl, and the fermentations broths were stirred for 60 minutes.

Protease activities in the supernatant were determined immediately after pH adjustment and after 60 minutes using the protease assay above. The protease concentrations were determined relative to the concentration in the Reference immediately after pH adjustment was set to 1. Results are shown in table 3

TABLE 3
Relative activities achieved:

| | T = 0 | T = 60 |
|---|---|---|
| Reference | 1 | 1.4 |
| 1HIS | 5.8 | 8.8 |
| 2HIS | 45 | 59 |
| 3HIS | 56 | 59 |
| 4HIS | 54 | 57 |
| 3IntHIS | 1.5 | 5 |

The results clearly shows that all the variants of the invention had higher solubility at low pH compared with the parent (=reference). The data also showed that even though the solubility for the variants of the invention were high from the start even more enzyme come into solution during the holding period of 60 minutes.

### Example 4

### Preparation and Expression of Variants

The following summarizes the mutation and introduction of an expression cassette into Bacillus licheniformis. All DNA manipulations were done by PCR (e.g. Sambrook et al.; Molecular Cloning; Cold Spring Harbor Laboratory Press) and can be repeated by everybody skilled in the art.

Two recombinant B. licheniformis strains were prepared. A reference strain was prepared by transforming the expression cassette encoding Savinase (SEQ ID NO: 2) into the Bacillus licheniformis host strain and selecting a transformant having 5 copies of the expression cassette with the Savinase gene integrated, and one strain having 5 copies of the expression cassette containing a modified gene encoding Savinase with 4 Histidine residues attached the N-terminus.

The recombinant organism and the reference organism were fermented in a standard lab fermenters and it was observed that the protein precipitated during the fermentations. The activities in the fermentations showed that the fermentations of Reference and 4HIS gave approximately same yield.

### Example 5

### Purification

The fermentation broths from Example 4 were diluted six fold with water and pH were adjusted to pH 4.5 at 40° C. using acetic acid, salt was added to control conductivity and the fermentations broths were stirred for 60 minutes in a water bath at 40° C.

Protease activities in the supernatant were determined immediately after pH adjustment and after 60 minutes using the protease assay above. The protease concentrations were determined relative to the concentration in the Reference immediately after pH adjustment was set to 1.

TABLE 4
Relative activities (%) achieved:

| | T = 0 | T = 60 |
|---|---|---|
| Reference (Savinase) | 19 | 60 |
| Savinase + 4HIS on N- | 63 | 87 |
| terminus | | |

The results clearly shows that using a recombinant microorganism having 5 copies of the modified gene led to a higher solubility at low pH compared with the reference recombinant microorganism transformed with only the gene of interest. The data also showed that even though the solubility for the variants of the invention were high from the start even more enzyme come into solution during the holding period of 60 minutes.

### Example 6

The following summarizes the mutation and introduction of an expression cassette into Bacillus licheniformis. All DNA manipulations were done by PCR (e.g. Sambrook et al.; Molecular Cloning; Cold Spring Harbor Laboratory Press) and can be repeated by everybody skilled in the art.

Two expression cassetes, one with gene encoding a Savinase variant (SEQ ID NO: 3) and one with a modified Savinase variant having the sequence of SEQ ID NO: 3, C-terminally extended with 4 Histidine residues (SEQ ID NO: 3+His-tag).

A recombinant strain was prepared by transforming the expression cassette encoding a Savinase variant (SEQ ID NO: 3) and the expression cassette containing the modified gene into the Bacillus licheniformis host strain and selecting a transformant having 5 copies of the expression cassette with the Savinase variant gene and one copy of the modified gene (extended with 4 His residues) integrated.

The recombinant organism was fermented in a standard lab fermenters and it was observed that the protein precipitated during the fermentations.

The fermentation broth was diluted six fold with water and pH were adjusted to pH 4.5 at 40° C. using acetic acid and the fermentations broths were stirred for 60 minutes in a water bath at 40° C.

Protease activities in the supernatant were determined immediately after pH adjustment and after 120 minutes using the protease assay above. The protease concentrations were determined relative to the concentration in the Reference immediately after pH adjustment was set to 1.

TABLE 5
Relative activities (%) achieved:

| | T = 0 | T = 15 | T = 30 | T = 60 | T = 120 |
|---|---|---|---|---|---|
| SEQ ID NO: 3 (5 copies) + | 48 | 68 | 89 | 93 | 84 |
| SEQ ID NO: 3 + His-tag | | | | | |

The results show that using the combination of 5 copies of the protease of interest (SEQ ID NO: 3) and one copy of a His-tagged version of the same protease led to a high and almost complete dissolution of the precipitated protease. In comparison, when the protease of interest is fermented alone (without a copy of the His-tagger version in the production strain) significantly less protease dissolves after lowering the pH to 4.5 and even after 120 minutes only a smaller fraction of the product dissolves under the tested conditions.

The results confirmed the benefits of obtaining a better solubility at low pH using a recombinant microorganism containing 5 copies of an expression cassette containing the protease of interest and one copy of the expression cassette containing the protease of interest having 4 Histidines attached to the C-terminus compared with a reference microorganism containing only the expression cassette containing the protease of interest.

### Example 7

The following summarizes the mutation and introduction of an expression cassette into Bacillus licheniformis. All DNA manipulations were done by PCR (e.g. Sambrook et al.; Molecular Cloning; Cold Spring Harbor Laboratory Press) and can be repeated by everybody skilled in the art.

Two expression cassetes, one with gene encoding a Savinase variant (SEQ ID NO: 3) and one with a modified Savinase variant having the sequence of SEQ ID NO: 4, C-terminally extended with 4 Histidine residues (SEQ ID NO: 4+His-tag).

A recombinant strain was prepared by transforming the expression cassette encoding a Savinase variant (SEQ ID NO: 4) and the expression cassette containing the modified gene into the Bacillus licheniformis host strain and selecting a transformant having 5 copies of the expression cassette with the Savinase variant gene and one copy of the modified gene (extended with 4 His residues) integrated.

The recombinant organism was fermented in a standard lab fermenters and it was observed that the protein precipitated during the fermentations.

The fermentation broth was diluted six fold with water and pH were adjusted to pH 4.5 at 40° C. using acetic acid and the fermentations broths were stirred for 60 minutes in a water bath at 40° C.

Protease activities in the supernatant were determined immediately after pH adjustment and after 120 minutes using the protease assay above. The protease concentrations were determined relative to the concentration in the Reference immediately after pH adjustment was set to 1.

TABLE 6
Relative activities (%) achieved:

| | T = 0 | T = 15 | T = 30 | T = 60 | T = 120 |
|---|---|---|---|---|---|
| SEQ ID NO: 4 (5 copies) + | 69 | 83 | 88 | 85 | 78 |
| SEQ ID NO: 4 + His-tag | | | | | |

The results show that using the combination of 5 copies of the protease of interest (SEQ ID NO: 3) and one copy of a His-tagged version of the same protease led to a high and almost complete dissolution of the precipitated protease. In comparison, when the protease of interest is fermented alone (without a copy of the His-tagger version in the production strain) significantly less protease dissolves after lowering the pH to 4.5 and even after 120 minutes only a smaller fraction of the product dissolves under the tested conditions.

The results confirmed the benefits of obtaining a better solubility at low pH using a recombinant microorganism containing 5 copies of an expression cassette containing the protease of interest and one copy of the expression cassette containing the protease of interest having 4 Histidines attached to the C-terminus compared with a reference microorganism containing only the expression cassette containing the protease of interest

### Example 8 Lysozyme Expression in A. Niger Strain

Construction of the Aspergillus Expression Cassette pJaL1470.

The expression plasmid pJaL1468 were made by amplification of the following 6 PCR fragments on 844 bp, 2972 bp, 3514 bp, 155 bp, 1548 bp and 2633 bp primer sets oJaL519 (GTTGTAAAACGACGGCCAGTTTCATCTTGAAGTTCCTA, SEQ ID NO: 5)/oJaL522 (CTGGCCGTCGTTTTAC, SEQ ID NO: 6), oJaL521 (GGATTTAGTCTTGATCGCGGCCGCACCATGCGTTTCATTTC, SEQ ID NO: 7)/oJaL524 (ATCAAGACTAAATCCTC, SEQ ID NO: 8), oJaL523 (TGGAAGTTACGCTCGCATTCTGTAAACGGGC, SEQ ID NO: 9)/oJaL526 (CGAGCGTAACTTCCACC, SEQ ID NO: 10), oJaL525 (GAGGGGATCGATGCGTCCGCGGGCGGAGAAGAAG, SEQ ID NO: 11)/oJaL528 (CGCATCGATCCCCTCGTC, SEQ ID NO: 12), oJaL527 (GATATCGGAGAAGCGTCCGCAGTTGATGAAGG, SEQ ID NO: 13)/oJaL530 (GCTTCTCCGATATCAAG, SEQ ID NO: 14) and oJaL529 (AGCTTGGCGTAATCATG, SEQ ID NO: 15)/oJaL520 (ACCATGATTACGCCAAGCTGCATGCATTAATTAACTTG, SEQ ID NO: 16), respectively, using plasmid pHUda1260 as template. The 6 PCR fragments were ligate together by Infusion cloning according to manufactory instruction and thereby creating plasmid pJaL1468.

For expression of an Acremonium alcalopphilum gene (SEQ ID NO: 17) encoding a GH25 lysozyme SEQ ID NO: 18 the coding region containing introns (SEQ ID NO: 17) was amplified as a PCR fragment on 878 bp (SEQ ID NO.: 19) by primer set oJaL513 (CAACTGGGGGCGGCCGCACCATGAAGCTTCTTCCCTCC, SEQ ID NO: 20) and oJaL514 (GTGTCAGTCACCGCGATCGCTTAGTCTCCGTTAGCGAG, SEQ ID NO: 21) using Acremonium alcalopphilum genomic DNA as template. The 878 bp PCR fragment was digested with AsiSII and NotII resulting in an 852 bp fragment. The 852 bp AsiSI-NotI fragment was cloned into the corresponding sites in pJaL1468, giving plasmid pJaL1470.

Construction of pHUda1260

The plasmid pHUda1260 was constructed by changing from the A. nidulans orotidine-5′-phosphate decarboxylase gene (pyrG) to the A. nidulans acetamidase gene (amdS) in pRika147.

Plasmid pRika147 (described in example 9 in WO2012160093) was digested with SphI and SpeI, and its ends were filled-in by use of T4 DNA polymerase followed by manufacture's protocol (NEB, New England Biolabs, Inc.). The fragment was purified by 0.8% agarose gel electrophoresis using TAE buffer, where a 9,241 bp fragment was excised from the gel and extracted using a QIAQUICK® Gel Extraction Kit.

Plasmid pHUda1019 (described in example 2 in WO2012160093) was digested with XbaI and Awl′, and its ends were filled-in by use of T4 DNA polymerase followed by manufacture's protocol (NEB, New England Biolabs, Inc.). The fragment was purified by 0.8% agarose gel electrophoresis using TAE buffer, where a 3,114 bp fragment containing amdS gene, A. oryzae tef1 (translation elongation factor 1) promoter and A. oryzae niaD (nitrate reductase) terminator was excised from the gel and extracted using a QIAQUICK® Gel Extraction Kit. The 9,241 bp fragment was ligated to the 3,114 bp fragment in a reaction composed of 1 μl of the 9,241 bp fragment, 3 μl of the 3,114 bp fragment, 1 μl of 5× ligase Buffer, 5 μl of 2× Ligase Buffer and 1 μl of Ligase (Roche Rapid DNA Ligation Kit). The ligation reaction was incubated at room temperature for 10 minutes. Five μl of the ligation mixture were transformed into DH5-alpha chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. One plasmid was designated as pHUda1260.

Construction of the Expression Plasmid pHiTe158

The 0.85 kb region of lysozyme gene from Acremonium alcalophilus was amplified from the plasmid pJaL1470 bp PCR with primer pairs HTJP-483 (agtcttgatcggatccaccatgaagcttcttccctccttg, SEQ ID NO: 22) and HTJP-504 (cgttatcgtacgcaccacgtgttagtggtggtggtggtctccgttagcgagagc, SEQ ID NO: 23).

The obtained 0.85 kb DNA fragment was ligated by In-Fusion® HD Cloning Kit (Clontech Laboratories, Inc) into the pHiTe50 (NZ 12683) digested with BamHI and PmlI to create pHiTe158.

### Transformation of Lysozyme Gene in A. Niger

Chromosomal insertion into A. niger (a derivative of NN059280 which is described in WO 2012/160093) of either native lysozyme (pJaL1470) or its variant with C-terminal tetra histidinetag gene (pHiTe158) with amdS selective marker was performed as described in WO 2012/160093. Strains which grew well were purified and subjected to southern blotting analysis to confirm whether the lysozyme gene in either pJaL1470 or pHiTe158 was introduced at NA1, NA2, SP288 or PAY loci correctly or not. The following set of primers to make non-radioactive probe was used to analyze the selected transformants.

### For Lysozyme Coding Region:

HTJP-483
(SEQ ID NO: 22)
Agtcttgatcggatccaccatgaagcttcttccctccttg
HTJP-513
(SEQ ID NO: 24)
Ctggtagcagtggtaggg

Genomic DNA extracted from the selected transformants was digested by SpeI and PmlI, then probed with lysozyme coding region. By the right gene introduction event, hybridized signals at the size of 5.1 kb (NA1), 1.9 kb (SP288), 3.1 kb (NA2) and 4.0 kb (PAY) by SpeI and PmlI digestion was observed probed described above.

Among the strains given the right integration events of 4-copies of the genes at NA1, NA2, SP288 and PAY loci, one strain with native lysozyme (1470-C3085-11) and one strain with the his-tagged variant (158-C3085-2) were selected.

### Example 9. Effect of Addition of Tetra Histidine Tag to the C-Terminal of Lysozyme on Protein Solubility

The strain with his-tagged lysozyme and the reference strain with native lysozyme gene were fermented in lab-scale tanks.

### Lab-Scale Tank Cultivation for Lysozyme Production

Fermentation was done as fed-batch fermentation (H. Pedersen 2000, Appl Microbiol Biotechnol, 53: 272-277). Selected strains were pre-cultured in liquid media then grown mycelia were transferred to the tanks for further cultivation of enzyme production. Cultivation was done at pH 4.75 at 34° C. for 7 days with the feeding of glucose and ammonium without over-dosing which prevents enzyme production. Culture broth and supernatant after centrifugation was used for enzyme assay

Crystal formation was observed in the fermentation broth for the native lysozyme but not in that for the His-tag form. Their enzyme activities (LSU activities) were measured followed by the methods described above; results are shown in the table 7 below.

TABLE 7

| | | LSU relative | LSU relative |
|---|---|---|---|
| | | activity in | activity in |
| Strain | Plasmids | whole broth | culture supernatant |
| 1470-C3085-11 | pJaL1470 | 1.00 | 0.26 |
| 158-C3085-2 | pHiTe158 | 1.35 | 1.50 |

Assumption: Density of culture broth is about 1 kg/L.

The LSU activity of the strains, wherein the lysozyme yields from the broth (prepared at 192 h of fermentation) in 1470-C3085-11 is normalized to 1.00. The insolubilized lysozyme (crystal) formed in 1470-C3085-11 during fermentation was (partially) solubilized by heat treatment at 50 C for 1 hour after dilution of the culture broth with water. The samples with no crystal in the transformants from pHiTe158 were equally treated.

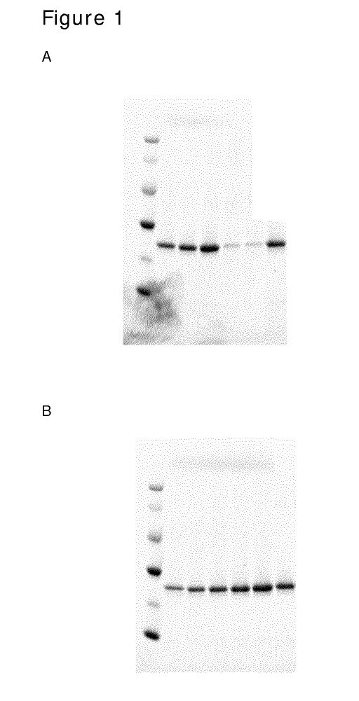

The supernatants samples from the fermentation of the A. niger strains were subjected to SDS-PAGE analysis to see and compare the degree of solubilization of the lysozyme. As anticipated, the lysozyme in the samples from 1470-C3085-11 significantly decreased throughout the fermentation due to crystallization (FIG. 1A, lanes 2-6, and table 8A) whereas those in the samples from 158-03085-2 continued to increase (FIG. 1B, lanes 2-6, and table 8A), suggesting that the solubility of lysozyme was strongly enhanced by the his-tag addition under the conditions.

TABLE 8A

| | | Fermentation time | LSU relative |
|---|---|---|---|
| Lane | Sample | (hours) | activity |
| 1 | Marker | | |
| 2 | 1470-C3085-11 | 97 hrs | 1.00 |
| 3 | 1470-C3085-11 | 120 hrs | 1.07 |
| 4 | 1470-C3085-11 | 144 hrs | 1.61 |
| 5 | 1470-C3085-11 | 169 hrs | 0.44 |
| 6 | 1470-C3085-11 | 192 hrs | 0.46 |
| 7 | Purified standard | | |

The LSU activity of the strains, wherein the lysozyme yields from the supernatant (prepared at 97 h of fermentation) in 1470-C3085-11 is normalized to 1.00.

TABLE 8B

| | | Fermentation time | LSU relative |
|---|---|---|---|
| Lane | Sample | (hours) | activity |
| 1 | Marker | | |
| 2 | 158-C3085-2 | 97 hrs | 1.00 |
| 3 | 158-C3085-2 | 120 hrs | 1.17 |
| 4 | 158-C3085-2 | 144 hrs | 1.74 |
| 5 | 158-C3085-2 | 169 hrs | 2.36 |
| 6 | 158-C3085-2 | 192 hrs | 3.02 |
| 7 | Purified standard | | |

The LSU activity of the strains, wherein the lysozyme yields from the supernatant (prepared at 97 h of fermentation) in 158-C3085-2 is normalized to 1.00.

1. A method for purifying a protein product, wherein at least part of the protein has 2-6 histidine residues located on the surface of the protein; in a process comprising the steps of:
a. Providing a fermentation broth,
b. Optionally adjusting the pH to a value below 6.0;
c. Optionally holding the mixture for a period; and
d. Separating the dissolved protein product from at least part of the solid materials from the fermentation broth.

2. The method of claim 1, wherein the 2-6 histidines located on the surface of the protein are located internally in the primary sequence, or wherein the 2-6 histidines located on the surface of the protein are in form of a C- and/or N-terminal extension of the protein.

3. The method of claim 2 where at least one of the 2-6 histidine residues at the surface of the protein of interest is provided by substitution or insertion.

4. The method of claim 1, wherein the protein product is an enzyme selected among hydrolase, isomerase, ligase, lyase, oxidoreductase, or transferase; such as an alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucano-transferase, glucoamylase, invertase, laccase, lipase, lysozyme, mannosidase, mutanase, oxidase, pectinolytic enzyme, peroxidase, phytase, polyphenoloxidase, proteolytic enzyme, ribonuclease, transglutaminase, or a xylanase.

5. The method of claim 4, wherein the enzyme is a protease having at least 80% sequence identity, preferably at least 90% sequence identity, preferably at least 95% sequence identity, preferably at least 96% sequence identity, preferably at least 97% sequence identity, preferably at least 98% sequence identity, preferably at least 99% sequence identity to SEQ ID NO: 1, SEQ ID NO: 2, SEQ ID NO: 3, SEQ ID NO: 4 or SEQ ID NO: 25.

6. The method of claim 4, wherein the enzyme is a lysozyme having at least 80% sequence identity, preferably at least 90% sequence identity, preferably at least 95% sequence identity, preferably at least 96% sequence identity, preferably at least 97% sequence identity, preferably at least 98% sequence identity, preferably at least 99% sequence identity to SEQ ID NO: 18.

7. The method of claim 1, wherein the solubility of the protein of interest at pH 4.5 is at least 10% higher than the solubility at pH 7.0, preferably at least 20% higher, preferably at least 30% higher, preferably at least 40% higher, preferably at least 50% higher, preferably at least 60% higher, preferably at least 70% higher, preferably at least 80% higher, preferably at least 90% higher, preferably at least 100% higher.

8. The method according to claim 1, wherein the concentration of the protein product in the fermentation broth is at least 3 g/l, such as at least 4 g/l, such as at least 5 g/l, such as at least 6 g/l; such as at least 7 g/l, such as at least 8 g/l, such as at least 9 g/l; such as at least 10 g/l, such as at least 11 g/l, such as at least 12 g/l; such as at least 13 g/l, such as at least 14 g/l, such as at least 15 g/l; such as at least 16 g/l, such as at least 17 g/l, such as at least 18 g/l; such as at least 19 g/l, such as at least 20 g/l.

9. The method according to claim 1, wherein the pH in step b) is adjusted to a pH value below, 6.0, preferably below 5.5, preferably below 5.0, preferably below 4.5.

10. The method according to claim 1, comprising a holding period in step c and wherein the holding period is in the range of 10 seconds to 90 minutes, preferably in the range of 1 minutes to 90 minutes, preferably in the range of 1 minute to 60 minutes, preferably in the range of 1 minutes to 30 minutes, such as in the range of 5 minutes to 30 minutes, and most preferred in the range of 10 to 20 minutes.

11. The method of claim 1, wherein the fermentation broth is provided by cultivating a microorganism in a growth medium.

12. The method of claim 11, wherein the microorganism is selected among bacteria, belonging to the genus Bacillus, such as Bacillus subtilis, B. lentus and B. lichiniformis, or among fungi belonging to the generi Aspergillus, Trichoderma, Penicillum, Fusarium, such as A. niger, A. awamori, A. oryzae, A. sojae, T. reesei, T. longibrachiatum or T. viride; or yeasts preferably belonging to the generi Saccharomyces, Pichia, Candida, Hanensula, Klyveromyces; such as S. cerevisiae, S. ovarum, P. Pastoris, K. lactis.

13. The method of claim 1, wherein the separation in step d is performed using filtration, centrifugation or decantation.

14. The method according to claim 1 wherein the method further comprises a pre-treatment step before the separation in step d, such as a dilution, salt addition and addition of a polymer.

15. The method of claim 1, wherein the protein product contains a protein of interest having a given amino acid sequence and a modified protein having same amino acid sequence except for a C- and/or N-terminal extension of 2-6 histidine residues.

16. The method of claim 1 comprising a holding period in step c and wherein the holding period is in the range of 10 seconds to 90 minutes, preferably in the range of 1 minutes to 90 minutes, preferably in the range of 1 minute to 60 minutes, preferably in the range of 1 minutes to 30 minutes, such as in the range of 5 minutes to 30 minutes, and most preferred in the range of 10 to 20 minutes.

17. A recombinant microorganism comprising at least one polynucleotide encoding an protein of interest operably linked to one or more control sequences that direct the production of the protein of interest and at least one polynucleotide encoding a modified protein, which in comparison with the protein of interest is modified to contain 2-6 histidine residues located on the surface of the protein, the modified gene operably linked to one or more control sequences that direct the production of the modified protein.

18. The recombinant microorganism of claim 17, which is a prokaryotic cell, preferably a Gram-positive cell, more preferably a Bacillus cell; most preferably a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis or Bacillus thuringiensis cell.

19. The recombinant microorganism of claim 17, which is an eukaryotic cell, preferably a fungal cell, more preferably an Aspergillus, Trichoderma or Saccharomyces or Pichia cell; most preferably an Aspergillus niger, Aspergillus oryzae, Aspergillus awamori, Aspergillus aculeatus, Trichoderma reesei, Trichoderma harzianum Trichoderma virede, Saccharomyces cerevisiae, Saccharomyces ovarum or Pichia pastoris cell.

20. The recombinant microorganism of claim 17, which comprises at least two copies of the polynucleotide encoding the protein of interest, preferably at least three copies, more preferably at least four copies and most preferably at least five copies of the polynucleotide encoding the protein of interest.

21. The recombinant microorganism of claim 17 which comprises at least two different polynucleotides encoding the same protein of interest, preferably at least three, more preferably at least four and most preferably at least five polynucleotides encoding the same protein of interest.

22. The recombinant microorganism according to claim 17, wherein the protein of interest is an enzyme, preferably a hydrolase, isomerase, ligase, lyase, oxidoreductase, or transferase, such as an alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucano-transferase, glucoamylase, invertase, laccase, lipase, lysozyme, mannosidase, mutanase, oxidase, pectinolytic enzyme, peroxidase, phytase, polyphenoloxidase, proteolytic enzyme, ribonuclease, transglutaminase, or a xylanase.

23. The recombinant microorganism of claim 22, wherein the enzyme is a protease; preferably the protease has an amino acid sequence with at least 80% sequence identity, preferably at least 90% sequence identity, preferably at least 95% sequence identity, preferably at least 96% sequence identity, preferably at least 97% sequence identity preferably at least 98% sequence identity, preferably at least 99% sequence identity to one of SEQ ID NO: 1, SEQ ID NO: 2, SEQ ID NO: 3, SEQ ID NO: 4 or SEQ ID NO: 25.

24. The recombinant microorganism of claim 22, wherein the enzyme is a lysozyme; preferably having an amino acid sequence with at least 80% sequence identity, preferably at least 90% sequence identity, preferably at least 95% sequence identity, preferably at least 96% sequence identity, preferably at least 97% sequence identity preferably at least 98% sequence identity, preferably at least 99% sequence identity to SEQ ID NO: 18.

25. The host cell of claim 17, wherein the polynucleotides are integrated into the chromosome of the host cell in different loci.

26. A method of producing an enzyme, said method comprising at step of cultivating a cell as defined in claim 17 under conditions conducive for production of the enzyme.

27. The method of claim 26, further comprising a step of recovering the enzyme.

28. A protein product comprising a protein of interest and a modified protein, which in comparison with the protein of interest is modified to have 2-6 histidine residues on the surface of the protein.

29. The protein product of claim 28, wherein the modified protein comprises 2-6 histidine residues attached to the C- and/or N-terminus of the protein.

30. The protein product of claim 28, wherein the protein of interest is an enzyme, preferably a hydrolase, isomerase, ligase, lyase, oxidoreductase, or transferase, such as an alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucano-transferase, glucoamylase, invertase, laccase, lipase, lysozyme, mannosidase, mutanase, oxidase, pectinolytic enzyme, peroxidase, phytase, polyphenoloxidase, proteolytic enzyme, ribonuclease, transglutaminase, or a xylanase.

31. The protein product of claim 30, wherein the enzyme is a protease; preferably the protease has an amino acid sequence with at least 80% sequence identity, preferably at least 90% sequence identity, preferably at least 95% sequence identity, preferably at least 96% sequence identity, preferably at least 97% sequence identity preferably at least 98% sequence identity, preferably at least 99% sequence identity to one of SEQ ID NO: 1, SEQ ID NO: 2, SEQ ID NO: 3, SEQ ID NO: 4 or SEQ ID NO: 25.

32. The protein product of claim 30, wherein the enzyme of interest is a lysozyme; preferably the lysozyme has an amino acid sequence with at least 80% sequence identity, preferably at least 90% sequence identity, preferably at least 95% sequence identity, preferably at least 96% sequence identity, preferably at least 97% sequence identity preferably at least 98% sequence identity, preferably at least 99% sequence identity to SEQ ID NO: 18.


SEQUENCE LISTING (25 sequences)

SEQ ID NO: 1 - protein, 270 aa, Artificial
  Variant protease
AQSVPWGISRVQAPAAHNRGLTGSGVKVAVLDTGIDSTHPDLNIRGGASFVPGEPSTQDGNGHGTHVAGTIAALDNSIGVLGVAPSAELYAVKVLGASGSGSVSSIAQGLEWAGNNGMDVANLSLGSPSPSATLEQAVNSATSRGVLVVAASGNSGAGSISYPARYANAMAVGATDQNNNRASFSQYGAELDIVAPGVNVQSTYPGSTYASLNGTSMATPHVAGAAALVLQKNPSWSNVQIRNHLKNTATSLGSTNLYGSGLVNAEAATR

SEQ ID NO: 2 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 3 - protein, 269 aa, artificial
  subtilisin variant
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSSATPHVAGAAALVKQKNPSWSNVQIRNHLKNTATSLGSTNLYGSGLVNAEAATR

SEQ ID NO: 4 - protein, 269 aa, Artificial
  Subtilase variant
AQSVPWGISRVQAPAAHNRGLTGSGVKVAVLDTGISTHPDLNIRGGASFVPGEPSTQDGNGHGTHAAGTIAALNNSIGVLGVAPSAELYAVKVLGASGSGSVSAIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 5 - DNA, 38 bp, Artificial
  Primer oJaL519
gttgtaaaacgacggccagtttcatcttgaagttccta

SEQ ID NO: 6 - DNA, 16 bp, Artificial
  Primer oLaL522
ctggccgtcgttttac

SEQ ID NO: 7 - DNA, 41 bp, Artificial
  Primer oJaL 521
ggatttagtcttgatcgcggccgcaccatgcgtttcatttc

SEQ ID NO: 8 - DNA, 17 bp, Artificial
  Primer oJaL524
atcaagactaaatcctc

SEQ ID NO: 9 - DNA, 31 bp, Artificial
  Primer oJaL 523
tggaagttacgctcgcattctgtaaacgggc

SEQ ID NO: 10 - DNA, 17 bp, Artificial
  Primer oJaL526
cgagcgtaacttccacc

SEQ ID NO: 11 - DNA, 34 bp, Artificial
  Primer oJaL525
gaggggatcgatgcgtccgcgggcggagaagaag

SEQ ID NO: 12 - DNA, 18 bp, Artificial
  Primer oJaL528
cgcatcgatcccctcgtc

SEQ ID NO: 13 - DNA, 32 bp, Artificial
  Primer oJaL527
gatatcggagaagcgtccgcagttgatgaagg

SEQ ID NO: 14 - DNA, 17 bp, Artificial
  Primer oJaL 530
gcttctccgatatcaag

SEQ ID NO: 15 - DNA, 17 bp, Artificial
  Primer oJaL 529
agcttggcgtaatcatg

SEQ ID NO: 16 - DNA, 38 bp, Artificial
  Primer oJaL 520
accatgattacgccaagctgcatgcattaattaacttg

SEQ ID NO: 17 - DNA, 838 bp, Acremonium alcalophilum
  CDS (1)..(147)
  Intron (148)..(301)
  CDS (302)..(835)
atgaagcttcttccctccttgattggcctggccagtctggcgtccctcgccgtcgcccggatccccggctttgacatttcgggctggcaaccgaccaccgactttgcaagggcgtatgctaatggagatcgtttcgtctacatcaaggtacgttcaaccttgccaccaagttgcgaacccgagacaagactgtgaccgcctcctttgccctggggcagctcacgcacccagcagcatcccatcccccggccccccacgtaccaccggaaagctaacatcaaccccctaccactgctaccaggccaccgagggcaccacattcaagagctccgcattcagccgccagtacaccggcgcaacgcaaaacggcttcatccgcggcgcctaccacttcgcccagcccgccgcgtcctcgggcgccgcgcaggcgagatacttcgccagcaacggcggcggctggtccaaggacggcatcaccctgcccggggcgctggacatcgagtacaaccccaacggcgccacctgctacggcctctcgcaatcggccatggtgaactggatcgaggactttgtcaccacctaccacggcatcacctcccgctggcccgtcatctacaccaccaccgactggtggacccagtgcaccggcaactccaaccgcttcgcgaaccgctgcccgctgtggatcgcccgctacgccagctccgtcggcactctgcccaatggctggggcttttacaccttctggcagtacaacgacaagtatcctcagggcggtgattcgaactggttcaacggcgatgcgtcgcgtctcagggctctcgctaacggagactaa

SEQ ID NO: 18 - protein, 227 aa, Acremonium alcalophilum
MKLLPSLIGLASLASLAVARIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 19 - DNA, 878 bp, Artificial
  PCR fragment
caactgggggcggccgcaccatgaagcttcttccctccttgattggcctggccagtctggcgtccctcgccgtcgcccggatccccggctttgacatttcgggctggcaaccgaccaccgactttgcaagggcgtatgctaatggagatcgtttcgtctacatcaaggtacgttcaaccttgccaccaagttgcgaacccgagacaagactgtgaccgcctcctttgccctggggcagctcacgcacccagcagcatcccatcccccggccccccacgtaccaccggaaagctaacatcaaccccctaccactgctaccaggccaccgagggcaccacattcaagagctccgcattcagccgccagtacaccggcgcaacgcaaaacggcttcatccgcggcgcctaccacttcgcccagcccgccgcgtcctcgggcgccgcgcaggcgagatacttcgccagcaacggcggcggctggtccaaggacggcatcaccctgcccggggcgctggacatcgagtacaaccccaacggcgccacctgctacggcctctcgcaatcggccatggtgaactggatcgaggactttgtcaccacctaccacggcatcacctcccgctggcccgtcatctacaccaccaccgactggtggacccagtgcaccggcaactccaaccgcttcgcgaaccgctgcccgctgtggatcgcccgctacgccagctccgtcggcactctgcccaatggctggggcttttacaccttctggcagtacaacgacaagtatcctcagggcggtgattcgaactggttcaacggcgatgcgtcgcgtctcagggctctcgctaacggagactaagcgatcgcggtgactgacac

SEQ ID NO: 20 - DNA, 38 bp, Artificial
  Primer oJaL513
caactgggggcggccgcaccatgaagcttcttccctcc

SEQ ID NO: 21 - DNA, 38 bp, Artificial
  Primer oJaL514
gtgtcagtcaccgcgatcgcttagtctccgttagcgag

SEQ ID NO: 22 - DNA, 40 bp, Artificial
  Primer HTJP-483
agtcttgatcggatccaccatgaagcttcttccctccttg

SEQ ID NO: 23 - DNA, 54 bp, Artificial
  Primer HTJP-504
cgttatcgtacgcaccacgtgttagtggtggtggtggtctccgttagcgagagc

SEQ ID NO: 24 - DNA, 18 bp, Artificial
  Primer HTJP-513
ctggtagcagtggtaggg

SEQ ID NO: 25 - protein, 275 aa, Bacillus amyloliquefaciens [sequence omitted]